(19) United States
(12) Patent Application Publication
(10) Pub. No.: US 2020/0088737 A1
(43) Pub. Date: Mar. 19, 2020

(54) Method for Detecting Glycoprotein

(71) Applicant: J-OIL MILLS, INC., Tokyo (JP)

(72) Inventors: Yuka KOBAYASHI, Tokyo (JP); Keiko SAITO, Tokyo (JP); Yasushi UENO, Tokyo (JP)

(21) Appl. No.: 16/070,751

(22) PCT Filed: Jan. 25, 2017
(86) PCT No.: PCT/JP2017/002517

§ 371 Date: Jul. 17, 2018

(30) Foreign Application Priority Data
Jan. 27, 2016 (JP) 2016-013188

(51) Int. Cl. G01N 33/68; C12Q 1/37; G01N 33/543

(52) U.S. Cl. CPC G01N 33/68; G01N 33/543; C12Q 1/37

(57) ABSTRACT

[Problem] To provide a method that increases the detection level of a reaction product generated through a reaction between a glycoprotein and a carbohydrate-binding compound in order to detect the glycoprotein with high precision.

[Solution] A method for detecting a glycoprotein according to the present invention comprising the steps of: subjecting a sample containing the glycoprotein to a protease treatment; allowing the protease-treated glycoprotein to react with a sugar-binding compound having affinity with a glycan contained in the glycoprotein in order to obtain a reaction product between the glycoprotein and the sugar-binding compound; and detecting the reaction product. The sugar-binding compound is preferably a sugar-binding protein.

### TECHNICAL FIELD

The present invention relates to a method for detecting glycoproteins, more specifically a method for detecting glycoproteins with improved detection sensitivity.

### BACKGROUND ART

More than half of proteins are glycosylated after translation. The manners of glycans binding to proteins are classified into an N-linked type in which a glycan binds to an amide group of an asparagine residue and an O-linked type in which a glycan binds to a hydroxyl group of a serine or threonine residue. Both glycosylations play important roles in protein activity, cell-cell interaction, adhesion and the like. There have been a lot of reports that change in glycosylation is associated with diseases.

For example, an α-fetoprotein (N-linked glycan) contained in serum scarcely exists in serum of healthy adults. On the other hand, in serum of a patient with benign liver disease, an α-fetoprotein-L1 (AFP-L1) type glycan increases, and furthermore, in a patient with liver cancer, α-fetoprotein-L3 (AFP-L3) type glycan is detected. Difference in the glycan detected with a lectin has been used for diagnosing liver diseases.

Haptoglobin is a glycoprotein having four N-linked glycan binding sites on a β chain. In pancreatic cancer, a lesional haptoglobin generated by addition of a fucose to haptoglobin is detected from serum and the like of a patient. The lesional haptoglobin increases with stage progression of pancreatic cancer and disappears after removal of a tumor part of pancreatic cancer. Early detection of pancreatic cancer is expected by precise and rapid detection of the fucosylated haptoglobin.

Thyroglobulin is a hormone that is synthesized in epithelial cells of a thyroid gland and accumulates in follicles, and generally has an activity of acting on cells all over the body to increase a cellular metabolic rate. A representative example of hyperthyroidism with excessively secreted thyroid hormone is Basedow's disease. Basedow's disease causes symptoms such as limb tremor, exophthalmos, palpitations, thyrocele, hyperhidrosis, weight loss, hyperglycemia and hypertension. Chronic thyroiditis (Hashimoto's disease) is an example of hypothyroidism with deficient thyroid hormone. Hashimoto's disease causes symptoms such as general malaise, hypohidrosis, weight gain and constipation. This protein has fucose, which is a kind of glycans. The measurement accuracy for the thyroglobulin content can be improved by enhancing detection sensitivity of a glycan added to a thyroglobulin.

In a patient with rheumatoid arthritis, an addition rate of terminal galactoses in serum IgG decreases and a rate of glycans having N-acetylglucosamine on their terminals increases. Galactose deletion markedly impairs important physiological functions of IgG: activation of complements and ability of binding to Fc receptors.

Transferrin (TF) is a glycoprotein having 679 amino acids, in which the 413rd and 611st aspartic acid residues are N-glycosylated with two branched glycans having sialic acids at terminals. The transferrin includes polymorphisms of TFC1 in which the 570th amino acid residue is proline and TFC2 in which the prolific is substituted with serine. In a patient with Alzheimer disease (AD) having a TFC1C2 heterozygotic genotype, a relative intensity of a TF having 6 sialic acids significantly decreases compared to that of patients having a TFC1C1 homozygotic genotype.

In a CSF glycoprotein collected from an AD patient, an addition rate of sialic acid significantly decreases. Changes in the amount of sialic acid have been observed for cardiovascular diseases, alcoholism, diabetes and the like, in addition to AD.

For detection of the glycoproteins, the use of a lectin that is one kind of sugar-binding compounds is known. The lectin is a generic name of proteins showing affinity with sugar residues such as sialic acid, galactose and N-acetylglucosamine. A large number of lectins derived from plants, animals or fungi having affinity with specific sugar residues have been discovered.

Lectin-ELISA (Enzyme-Linked Immunosorbent Assay) and lectin affinity chromatography is known as a method for detecting a glycoprotein using a lectin. The lectin-ELISA has advantages such as an ability of simultaneously measuring a large number of specimens and an ability of relatively easily measuring glycans. The lectin affinity chromatography utilizes a property of lectin to bind specifically to glycans, distinguish slight differences between glycan structures and separate them. The lectin affinity chromatography, in which a lectin HPLC column for high-performance liquid chromatography (1-IPCL) is used), is effective not only for analyzing but also purifying glycans.

### PRIOR ART DOCUMENTS

### Patent Documents

Patent Document 1: JP Pat. No. 4514163 (fucose α1→6 specific lectin)

### Non-Patent Documents

Non-Patent Document 1: Yuka Kobayashi et al., “A Novel Core Fucose-specific Lectin from the Mushroom Pholiota squarrosa”, J. Biol. Chem, 2012, 287, p 33973-33982
Non-Patent Document 2: Naoto Shibuya et al., “The Elderberry (Sambucus nigra L.) Bark Lectin Recognizes the Neu5Ac(a2-6)Gal/GalNAc Sequence”, J. Biol. Chem, 1987, 262, p 1596-1601
Non-Patent Document 3: T. B N G et al., “Isolation and characterization of a galactose binding lectin with insulinomimetic activities”, Int. J. Peptide Protein Res 28, 1986, p 163-173
Non-Patent Document 4: Yasuo Oda et al., “A new agglutinin from the Tulipa gesneriana bulbs”, Eur. J. Biochem, 1087, 165, p 297-302

### SUMMARY OF INVENTION

### Problem to be Solved

In the glycoprotein detection method such as the lectin ELISA and the lectin affinity chromatography, if a signal due to the reaction between a glycoprotein and a lectin is low, it is difficult to precisely detect the dycoprotein. An increased signal attributed to the lectin reaction is desirable for early and precisely diagnosing a disease associated with change in an amount of glycans.

Thus, an object of the present invention is to provide a method for increasing a signal (reaction value) attributed to a reactant between a glycoprotein and a sugar-binding compound including a lectin in order to precisely detect glycoproteins.

### Solution to Problem

As a result of intensive studies on the above problems, the present inventors have found that the above problems can be solved by subjecting the glycoprotein to a protease treatment before the reaction with the sugar-binding compound. That is, the present invention provides a method for detecting glycoproteins, comprising steps of:

subjecting a sample containing the glycoprotein to a protease treatment; and

allowing the protease-treated glycoprotein to react with a sugar-binding compound having affinity with a glycan contained in the glycoprotein in order to detect a reactant between the glycoprotein and the sugar-binding compound.

The term “glycoprotein” is herein used to include a glycopeptide. The term “glycan” is herein used to include a monosaccharide. In addition, the “sugar-binding compound” herein means a compound capable of binding to a sugar. The “sugar-binding compound” is preferably a sugar-binding protein.

The protease treatment is a pepsin treatment, papain treatment or an actinase treatment, for example.

The sample is serum, for example.

The sugar-binding compound has affinity with at least one selected from the group consisting of fucose, sialic acid, mannose, glucose, galactose, N-acetylglucosamine and N-acetylgalactosamine.

Preferably, the glycoprotein is immobilized to a support.

Preferably, the glycoprotein is immobilized to the support via its antibody.

Preferably, the sugar-binding compound and/or a probe for detecting the sugar-binding compound are labeled.

The glycan is glycan of complex type, a glycan of high mannose type or an O-linked glycan, for example.

The glycoprotein is selected from a group consisting of haptoglobin, fucosylated haptoglobin, transferrin, γ-glutamyltranspeptidase, immunoglobulin G, immunoglobulin A, immunoglobulin M, α1-acidic glycoprotein, α-fetoprotein, fucosylated α-fetoprotein, fibrinogen, human placenta chorionic gonadotropin, carcinoembryonic antigen, prostate-specific antigen, fucosylated prostate-specific antigen, thyroglobulin, fetuin and asialofetuin, for example.

### Effects of Invention

According to the glycoprotein detection method of the present invention, a signal (reaction value) attributed to a reactant between a protease-treated glycoprotein and a sugar-binding compound increases compared to a protease-untreated glycoprotein. For glycoproteins for which deletion or addition of a glycan is known to be associated with a disease, an increased signal leads to early detection, diagnosis and treatment of the disease. Also, it is expected to be useful in elucidation of pathogenic mechanisms of diseases, and medical and biochemical studies on treatment and prevention.

### DESCRIPTION OF EMBODIMENTS

Hereinafter, an embodiment of the present invention will be described in detail. The glycoprotein assay method of the present invention comprises: subjecting a sample containing a glycoprotein to a protease treatment; allowing the protease-treated glycoprotein to react with a sugar-binding compound having affinity with a glycan contained in the glycoprotein in order to obtain a reactant between the glycoprotein and the sugar-binding compound; and detecting the reactant. The method of the present invention is the same as the conventional glycoprotein detection method using a sugar-binding compound, except for that the method of the present invention essentially comprises the step of subjecting the sample containing the glycoprotein to the protease treatment.

The glycoproteins to be measured in the present invention is not particularly limited as long as they have glycans. The glycans include an N-linked glycan and an O-linked glycan. The N-linked glycan includes:

a conjugated glycan in which 1 to 6 side chains composed of fucose, sialic acid, galactose and N-acetylglucosamine (N-acetyllactosamine structure, poly N-acetyllactosamine structure) are added to a core structure represented by the following formula:

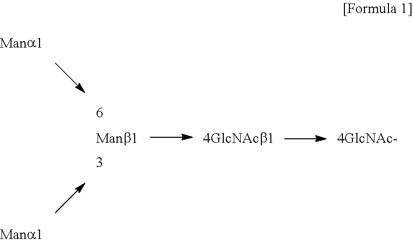

[wherein, Man refers to mannose, and GlcNAc refers to N-acetyllactosamine];

a high mannose type glycan in which an oligosaccharide composed only of mannose is added to the core structure; and

a hybrid type glycan in which the conjugated type and the high mannose type are mixed.

Also, the N-linked glycan includes a glycan in which fucose is added to the N-acetylglucosamine at the reducing terminal of the core structure.

The sugar residues to be detected in the present invention include sialic acid (Sia), galactose (Gal), mannose (Man), glucose (Glc), N-acetylglucosamine (GlcNAc), N-acetylgalactosamine (GalNAc), fucose (Fuc), and the like. These sugar residues may be added to or deleted from a glycan structure which a healthy person normally has.

Specific examples of the glycoproteins include haptoglobin (HP), fucosylated haptoglobin (fHP), transferrin (IF), γ-glutamyltranspeptidase (γ-GTP), immunoglobulin G (IgG), immunoglobulin A (IgA), immunoglobulin M (IgM), α1-acidic glycoprotein, α-fetoprotein (AFP), fucosylated (t-fetoprotein (fAFP, AFP-L3), fibrinogen, hCG (human placenta chorionic gonadotropin), CEA (carcinoembryonic antigen), prostate-specific antigen (PSA), fucosylated prostate-specific antigen (fPSA), thyroglobulin (TG), fetuin (FET), asialofetuin (aFET) and the like. A glycoprotein for which a relationship between a change in a glycan structure in the glycoprotein and a disease or abnormality is suggested is preferable.

The origin of the samples containing the glycoprotein is not particularly limited. The examples thereof include blood, plasma, serum, tear, saliva, body fluid, milk, urine, culture supernatant of cells, secretion from a transgenic animal, and the like. Blood, plasma or serum is preferable, and serum is particularly preferable. For the protease treatment, the samples containing the glycoprotein such as serum may be diluted in advance.

In the method of the present invention, the glycoprotein is treated with a protease (proteolytic enzyme) before the reaction with the sugar-binding compound. The protease is not particularly limited as long as it acts on a glycoprotein to produce a glycopeptide. The proteases can be functionally classified into aspartic protease (acid protease), serine protease, cysteine protease, metalloprotease, N-terminal threonine protease, glutamic protease and the like.

The protease may be originated from anything. The protease includes: animal-derived proteases such as pepsin, trypsin, chymotrypsin, elastase, cathepsin D and calpain; plant-derived proteases such as papain, chymopapain, actinidin, kallikrein, ficin and bromelain; and microorganism-derived proteases such as those derived from Bacillus, Aspergillus, Rhizopus, blue mold (Penicillium), Streptomyces, Staphylococcus, Clostridium and Lysobacter.

As the protease used in the present invention, a commercial product can be used without particular limitation. For example, the pepsin includes a swine gastric mucosa-derived pepsin (from Sigma-Aldrich Co. LLC), the papain includes a papaya-derived papain (from Funakoshi Co., Ltd.), and the Streptomyces-derived protease includes Actinase E (from Laken Pharmaceutical Co., Ltd.) and the like.

The amount of the protease for use may be any amount allowing progression of the reaction of the glycoprotein with the protease. The concentration thereof during the reaction may be normally 0.0001 to 5 mg/mL. The pepsin is particularly preferred because it is effective in only a slight amount such as 0.0001 to 1 mg/mL.

The conditions such as pH, temperature, and time for the protease treatment depend on the kind of the proteolytic enzymes for use. The protease treatment using a pepsin is carried out at pH of normally 1.5 to 5, preferably pH 2 to 4; temperature of normally 10 to 60° C., preferably 15 to 45° C., more preferably 20 to 40° C., for a period of normally 1 minute to 24 hours, preferably 1 minute to 180 minutes, more preferably 2 minutes to 120 minutes, still more preferably 2 minutes to 60 minutes. The protease treatment using a papain is carried out at pH of normally 3 to 10, preferably pH 5 to 8; temperature of normally 20 to 80° C., preferably 20 to 45° C., more preferably 20 to 40 CC, for a period of normally 1 minute to 24 hours, preferably 1 minute to 180 minutes, more preferably 2 minutes to 120 minutes, still more preferably 2 minutes to 60 minutes. The protease treatment using an Actinase E is carried out at pH of normally 7 to 10, preferably pH 7 to 8.5; temperature of normally 10 to 50° C., preferably 20 to 45° C., more preferably 20 to 40 CC, for a period of normally 1 minute to 24 hours, preferably 1 minute to 180 minutes, more preferably 2 minutes to 120 minutes, still more preferably 2 minutes to 60 minutes.

After the protease treatment, the enzymatic reaction is terminated by an appropriate means such as change of pH, heat treatment and addition of an enzymatic reaction-terminating liquid. Subsequently, the reaction solution may be separated into a supernatant and a solid residue by a separation means such as filtration, dialysis and centrifugation.

The protease-treated glycoprotein is reacted with a sugar-binding compound having affinity with a glycan contained in the glycoprotein to obtain a reactant between the glycoprotein and the sugar-binding compound.

During the reaction between the protease-treated glycoprotein and the sugar-binding compound, the glycoprotein or the sugar-binding compound do not necessarily need to be immobilized, but they are preferably immobilized. Examples of the support for immobilizing the glycoprotein include beads, a disk, a stick, a tube, a microtiter plate, a microsensor chip, a microarray, and the like which are made of materials such as glass, polyethylene, polypropylene, polyvinyl acetate, polyvinyl chloride, polymethacrylate, latex, agarose, cellulose, dextran, starch, dextrin, silica gel and porous ceramics.

As a method for immobilizing glycoproteins on the support, a general-purpose method such as physical adsorption, covalent binding and crosslinking can be used without particular limitation.

The glycoprotein may be immobilized on a support via its antibody. The antibody may be the antibody molecule itself, or may be an active fragment containing an antigen-recognition site such as Fab, Fab′, F(ab′ obtained by enzymatic treatment of the antibody.

The origin of the antibody is not limited. The antibodies include an antisera and an ascites fluid obtained by immunizing a mammal such as human, mouse and rabbit with a glycoprotein as an antigen, as well as a polyclonal antibody obtained by purifying them by a general-purpose method such as salting-out, gel filtration, ion exchange chromatography, electrophoresis and affinity chromatography. Furthermore, the antibody include a monoclonal antibody obtained by a process that an antibody-producing lymphocyte of a mouse immunized with a protein prepared from a human or animal serum or the like is fused with a myeloma cell to obtain a hybridoma that produces a monoclonal antibody capable of recognizing the glycoprotein, then the hybridoma or a cell line derived therefrom is cultured, and the monoclonal antibody is collected from the culture. For general-purpose glycoproteins, antibodies thereof are sold as reagents, and they can be used without limitation in the present invention.

When the above-described antibody or the like has a glycan capable of reacting with a sugar-binding compound, the glycan is removed from the antibody as appropriate. Methods for obtaining an antibody having no glycan capable of reacting with a sugar-binding compound include: a method of treating a monoclonal antibody with a glycan degrading enzyme such as neuraminidase, β-galactosidase and N-glycanase; a method of subjecting an Fc portion of an antibody to limited proteolysis with a proteolytic enzyme such as pepsin and papain; a method of oxidatively decomposing a glycan structure with a periodic acid aqueous solution; and a method of adding a glycan synthesis inhibitor to a medium of a hybridoma or an animal cell derived from the hybridoma and culturing the medium.

As a method for immobilizing the antibody to the support, general-purpose methods such as physical adsorption, covalent binding and crosslinking can be used without particular limitation. A solution of an antibody against a glycoprotein (e.g. anti-transferrin antibody) is added to the support to bind the antibody to the support.

A solution of the glycoprotein is added to the support to which the antibody binds, whereby binding the glycoprotein due to the antigen-antibody reaction.

The term “sugar-binding compound” means a compound having affinity with a glycan contained in a glycoprotein. The sugar-binding compound for use is appropriately selected depending on the glycan capable of binding to the glycoprotein.

The sugar-binding compound is e.g., a protein (including peptide) capable of binding to a sugar, as well as a nucleic acid such as DNA and RNA capable of binding to a sugar.

Examples of the sugar-binding protein include lectin, anti-glycan antibody, maltose binding protein, glucose binding protein, galactose binding protein, cellulose binding protein, chitin binding protein and sugar-binding module. The sugar-binding compound is preferably a sugar-binding protein, more preferably lectin and an anti-glycan antibody, still more preferably lectin.

The sugar-binding compounds may be used either alone or in combination of two or more kinds.

When the affinity of the lectin is expressed as the minimum inhibitory concentration of the sugar capable of inhibiting hemagglutination, it is normally 100 mM or less, preferably 10 mM or less. The minimum inhibitory concentration means the minimum concentration required for the sugar to inhibit the agglutination, indicating that the less the minimum inhibitory concentration is, the more the affinity with the lectin is. The hemagglutination inhibition test method can be carried out in accordance with the method described in Patent Document 1 (Japanese Patent No. 4514163). Japanese Patent No. 4514163 is incorporated herein for reference.

The lectin may be either a naturally-derived lectin or a lectin obtained by chemical synthesis or genetic engineering synthesis. The natural lectin may be originated from any of a plant, an animal and a fungus. Examples of natural lectins that can be used in the present invention are shown below.

Examples of lectins having affinity with galactose (Gal)/N-acetylgalactosamine (GalNAc) include Agaricus bisporus lectin (ABA), Dolichos biflorus lectin (DBA), Erythrina cristagalli lectin (ECA), Phaseolus vulgaris lectin (PHA-E4, PHA-P), peanut lectin (PNA), soybean lectin (SBA), Bauhinia purpurea lectin (BPL) and Ricinus communis lectin (RCA 120). Examples of lectins having affinity with mannose (Man)/glucose (Glc) include concanavalin A (ConA), Lens culinaris lectin (LCA-A) and Pisum sativum lectin (PSA). Examples of lectins having affinity with fucose (Fuc) include Aleuria aurantia lectin (AAL), Lens culinaris lectin (LCA, LCA-A), lotus lectin (Lotus), Pisum sativum lectin (PSA), Ulex europaeus lectin (UEA), Lotus corniculatus lectin (LTA), Narcissus pseudonarcissus lectin (NPA), Vicia faba lectin (VFA), Aspergillus oryzae lectin (AOL), Pholiota squarrosa lectin (PhoSL), Pholiota terrestris lectin (PTL), Stropharia rugosoannulata lectin (SRL), Naematoloma sublateritium lectin (NSL), Lepista sordida lectin (LSL) and Amanita muscaria lectin (AML). Above all, the PhoSL, PTL. SRL, NSL, LSL and AML are advantageous for detecting a glycoprotein for which the addition or deletion of the α→6 fucose is associated with diseases, because they specifically bind to the α→4; fucose. Examples of lectins having affinity with N-acetylglucosamine (GlcNAc) include Datura stramonium lectin (DSA), pokeweed lectin (PWM), wheat germ lectin (WGA), Griffonia simplicifolia lectin-II (GSL-II) and Psathyrella velutina Lectin (PVL). Examples of lectins having affinity with sialic acid (Sia) include Maackia amurensis lectin (MAM), Sambucus sieboldiana lectin (SSA), wheat germ lectin (WGA), Agrocybe cylindracea lectin (ACG), Trichosanthes japonica lectin (TJA-I), Psathyrella velutina lectin (PVL), Sambucus nigra lectin (SNA-1) and the like.

The sugar-binding compound is preferably labeled with a labeling means known in the art. Also, the sugar-binding compound may be detected via a probe capable of reacting with the sugar-binding compound (e.g., an antibody capable of binding the sugar-binding compound such as an anti-lectin antibody). In this case, the probe is preferably labeled with a labeling means known in the art. The probe for detecting the sugar-binding compound may be used either alone or in combination of two or more kinds.

Examples of the labeling means of the sugar-binding compound or the probe for detecting the sugar-binding compound may include: an enzyme such as horseradish peroxidase (HRP), alkaline phosphatase (AP), β-D-galactosidase, glucose oxidase and glucose-6-phosphate dehydrogenase; a fluorescent compound such as fluorescein isothiocyanate (FITC), tetramethylrhodamine B isothiocyanate (TRITC), rhodamine and CyDye; a radioactive substance such as ¹²⁵I, ³H and ¹⁴C; a metal colloid such as gold sol, silver sol and platinum sol; a synthetic latex such as polystyrene latex colored with a pigment; biotin; and digoxigenin.

When the labeling means is an enzyme, a chromogenic substrate is used to measure an enzyme activity. The substrate for the horseradish peroxidase (HRP) includes 3,3′,5,5′-tetramethylbenzidine (TMB), 2,2′-azino-di-[3-ethylbenzthiazoline sulfonic acid] diammonium salt, 5-aminosalicylic acid, or o-phenylenediamine (OPD). The substrate for the alkaline phosphatase includes p-nitrophenyl phosphate (PNPP) or 4-methylumbelliferyl phosphate. The substrate for the β-D-galactosidase is exemplified by o-nitrophenol-β-D-galactopyranoside.

The labeling means can be bound to the sugar-binding compound or the probe for detecting the sugar-binding compound in accordance with a conventional method. In particular, bonding the label via a streptavidin (or avidin)-biotin system is preferable from the viewpoint of increasing the sensitivity.

The glycoprotein is appropriately exposed to a solution containing the sugar-binding compound in order to react the immobilized glycoprotein with the sugar-binding compound.

After the reaction with the sugar-binding compound, a reactant between the glycoprotein and the sugar-binding compound is detected. The detection method of the reactant between the glycoprotein and the sugar-binding compound is not particularly limited, and a method well known to those skilled in the art can be used. Examples of the detection method include: a method for detecting color development, luminescence, fluorescence of an enzyme or the like, such as lectin ELISA (direct adsorption method, sandwich method), lectin affinity chromatography (using, for example, an HPLC-lectin column), lectin-affinitive electrophoresis and lectin staining; a method for detecting evanescent waves, such as glycan arrays and lectin arrays; a method for detecting a mass change, such as a crystal oscillator microbalance method and a surface plasmon resonance method; and the like. The surface plasmon resonance method is convenient because a mass of the glycoprotein immobilized on a support and an amount of a detected sugar-binding compound bound to a glycoprotein can be simultaneously measured by a multistage approach.

The amount of glycans, which is expressed by an amount of a marker (absorbance etc.) in the labeled sugar-binding compound and the like, can be compared with that in a reference sample in order to suspect the change thereof. For example, the glycoprotein concentration of a specimen is adjusted to a certain value, the measurement result of the detected sugar-binding component would reflect the change of an amount of a specific glycan added to the glycoprotein. A disease associated to glycosylation changes of protein can be diagnosed more precisely than the conventional methods by comparing a healthy person and a patient for the amount of the glycans in the glycoprotein,

Quantification of the rate of change in an amount of sugar residues (the degree of addition or deletion of sugars) serves well for the diagnosis and treatment as well as the prevention, study and the like of diseases associated to the amount of glycosylation changes. In order to acquire an addition rate of sugars, a total amount of sugar residues in a reference sample (e.g., a glycoprotein of a healthy person, or a commercial or synthesized reagent) to which a target sugar residue is added on every place in glycan terminals is measured in advance by the detection method of the present invention. The amount of sugar residues is expressed by an amount of a marker (absorbance etc.) in the labeled detected sugar-binding compound. Subsequently, an amount of sugar residues of an unknown sample is measured by the detection method of the present invention. A value obtained by dividing the amount of sugar residues measured for the unknown sample by the total amount of sugar residues is taken as an addition rate of sugars. A value obtained by subtracting the addition rate of sugar from 1 is taken as a deletion rate of sugars.

The rate of change in an amount of glycans may be represented by a ratio of an amount of glycans with respect to an amount of a glycoprotein in a sample to be measured. The amount of the glycoprotein is first acquired by absorbance, Enzyme-Linked. Immunosorbent Assay, Bradford method, Lowry method or the like.

It is advantageous in terms of the efficiency of the measurement operation to utilize a calibration curve in derivation of the addition or deletion rate of sugars. A plurality of standard samples with known target amounts of sugar residues and different amounts of sugar residues are measured by the detection method of the present invention in order to acquire a relationship between the target amount of sugar residues and an amount of a marker (absorbance etc.) in the detected sugar-binding compound, and on the basis of this relationship, a calibration curve is made in advance. An amount of a marker (absorbance etc.) in a sample with an unknown target amount of sugar residues is acquired by the detection method of the present invention, and the amount of the marker is fitted to the calibration curve.

Several representative detection methods will be roughly explained below. In the ELISA (direct adsorption method), a specimen such as serum containing a glycoprotein is added to an ELISA plate and immobilized (specimen reaction), Subsequently, a biotin-labeled lectin is added to react the glycan with the lectin (lectin reaction, primary reaction). An HRP-labeled streptavidin solution is added as a secondary labeled compound to react the biotin with the streptavidin (probe reaction, secondary reaction). Subsequently, a chromogenic substrate for the HRP is added to develop color, and a coloring intensity is measured with an absorptiometer. A calibration curve is previously graphed with a standard sample containing a known concentration of glycans, so that the glycan can also be quantitatively determined.

In the ELISA (sandwich method), an antibody capable of binding to a glycoprotein (antigen) is added to an ELISA plate, and the antibody is immobilized on the plate. Subsequently, a specimen containing a glycoprotein (such as serum) is added to react the antibody with the glycoprotein (specimen reaction). Subsequently, a biotin-labeled lectin is added to react the glycan with the lectin (lectin reaction). An HRP-labeled streptavidin solution is added as a secondary probe to react the biotin with the streptavidin (probe reaction). Subsequently, a chromogenic substrate for the HRP is added to develop color, and a coloring intensity is measured with an absorptiometer. A calibration curve is previously graphed with a known concentration of standard sample, so that the glycan can also be quantitatively determined.

In accordance with the method of the present invention, the detection sensitivity for the glycoprotein is improved, contributing to improvement of the diagnostic accuracy for diseases associated with change of the glycan. Examples of diseases for which a galactose residue may be a diagnostic indicator include chronic rheumatoid arthritis, liver cancer, myeloma and the like. Examples of diseases for which a mannose residue may be a diagnostic indicator include rectal cancer and the like. Examples of diseases for which a fucose residue may be a diagnostic indicator include colon cancer, pancreatic cancer, liver cancer and the like. Examples of diseases for which an N-acetylglucosamine residue may be a diagnostic indicator include idiopathic normal pressure hydrocephalus, liver cancer and the like. Examples of diseases for which a sialic acid residue may be a diagnostic indicator include Alzheimer's disease, cardiovascular disease, alcoholism, IgA nephropathy, liver cancer, prostate cancer, ovarian cancer, myocardial infarction, fibrosis, pancreatitis, diabetes, glycoprotein-glycan transfer deficiency, and the like.

### EXAMPLES

Hereinafter, the present invention will be described in more detail with reference to Examples of the present invention. However, the present invention is not limited to the following Examples.

The reagents for use in the present invention were prepared as shown below.

### (1) Buffer Solution

### [Phosphate Buffered Saline (PBS)]

5.75 g of disodium hydrogenphosphate (from Wako Pure Chemical Corp.), 1.0 g of potassium dihydrogenphosphate (from Wako Pure Chemical Corp.), 1.0 g of potassium chloride (from Wako Pure Chemical Corp.) and 40.0 g of sodium chloride (from Wako Pure Chemical Corp.) were weighed, and the volume was further increased to 5 L with water. The resulting solution was to be phosphate buffered saline (PBS).

### [0.05% Tween/PBS]

2.5 mL of polyoxyethylene (20) sorbitan monolaurate (trade name: Tween 20, from Nacalai Tesque Co., Ltd.) was dissolved in 51, of PBS to obtain a PBS solution of 0.05% Tween 20 (hereinafter referred to as 0.05% Tween/PBS).

[1 M glycine-Hydrochloric Acid Buffer (pH 3.0)]

75.07 g of glycine (from Wako Pure Chemical Corp.) was dissolved in about 900 mL of water, to which 6N hydrochloric acid was added to adjust the pH level to 3.0 with a pH meter, and in addition, the volume was further increased to 1000 mL with water.

### [0.5 M Glycine-Hydrochloric Acid Buffer (pH 3.3)]

37.54 g of glycine described above was dissolved in about 900 mL of water, to which 6N hydrochloric acid was added to adjust the pH level to 3.3 with a pH meter, and in addition, the volume was further increased to 1000 ml with water.

### [1 M Tris-Hydrochloric Acid Buffer (pH 9.0)]

60.6 g of tris-hydroxymethylaminomethane (from Nacalai Tesque Co., Ltd.) was dissolved in about 400 mL of water, to which 6 N hydrochloric acid was added to adjust the pH level to 9.0 with a pH meter, and the volume was further increased to 500 mL with water.

### [0.33 M Tris-Hydrochloric Acid Buffer (pH 9.0)]

20.2 g of tris-hydroxymethylaminomethane described above was dissolved in about 400 mL of water, to which 6 N hydrochloric acid was added to adjust the pH level to 9.0 with a pH meter, and the volume was further increased to 500 mL with water.

### [10 mM Calcium Chloride/100 mM Tris-Hydrochloric Acid Buffer (pH 7.8)]

0.74 g of calcium chloride (from Wako Pure Chemical Corp.) was weight out into a 5 mL tube and dissolved in 5 mL of water to obtain a 1 M calcium chloride solution. 12.11 g of tris-hydroxymethylaminomethane described above was weighed out and dissolved in 80 mL of water, to which 6 N hydrochloric acid solution was added to adjust the pH level to 7.8, and the liquid volume was further increased to 100 mL with water to obtain a 1 M tris-hydrochloric acid buffer (pH 7.8). A 10 mM calcium chloride/100 mM tris-hydrochloric acid buffer (pH 7.8) was prepared from the 1 M calcium chloride solution and the 1 M tris-hydrochloric acid buffer (pH 7.8).

### (2) Glycoprotein Solution

Each of the following glycoproteins was dissolved in water so that the concentration was 2 mg/mL, to obtain a glycoprotein solution.

Transferrin (TF): from Sigma-Aldrich Co. LLC.
Immunoglobulin G (IgG): from Sigma-Aldrich Co, LLC.
α-fetoprotein (AFP): from Funakoshi Co., Ltd.
α-fetoprotein-L3 (AFP-L3): prepared in accordance with the method described in Non-patent document 1.

### (3) Protease Solution

A protease solution shown below was prepared.

### [Pepsin]

A pepsin (derived from a swine gastric mucosa, from Sigma-Aldrich Co. LLC, Code No. P6887) was dissolved in a 1 M glycine-hydrochloric acid buffer (pH 3.0) so that the concentration was 0.1 mg/mL (0.01 mass %).

### [Papain]

A papain (derived from a papaya, from Funakoshi Co., Ltd., Code No. LS003126) was dissolved in a cysteine solution (1.1 mM EDTA, 0.2 M phosphate buffer containing 0.067 mM cysteine hydrochloride, pH 6.5) so that the concentration was 2 mg/mL (0.2 mass %).

### [Actinase E]

An Actinase E (from Kaken Pharmaceutical Co., Ltd., Code No. 90002-1611) was dissolved in a 10 mM calcium chloride/100 mM tris-hydrochloric acid buffer (pH 7.8) so that the concentration was 10 mg/mL (1 mass %).

### (4) PMSF Solution

17.4 mg of phenylmethylsulfonyl fluoride (PMSF, from Wako Pure Chemical Corp.) was dissolved in 500 μL of dimethylsulfoxide (DMSO, from Wako Pure Chemical Corp.) to prepare a 200 mM PMSF solution. This solution was diluted with water by 20 times immediately before use to prepare a 10 mM PMSF solution.

### (5) BSA/PBS

1 g of bovine serum albumin (BSA, from Sigma-Aldrich Co. LLC) was dissolved in 100 mL of PBS to prepare a PBS solution of BSA (BSA concentration of 1%).

### (6) Lectin

A lectin shown in Table 1 was prepared. A PhoSL, which is an α1→6 fucose specific lectin, was purified from Pholiota squarrosa in accordance with the method described in Non-patent document 1. A synthesized PhoSL peptide (SEQ ID No. 1), which is a kind of synthesized peptides of PhoSL, was synthesized in accordance with the method described in Non-patent document 1. Aspergillus orvzae lectin (AOL) was obtained from Tokyo Chemical Industry Co., Ltd. Biotin-labeled lectins from J-OIL MILLS Inc. were used as Sambucus nigra lectin (SNA-I), Momodica charantia lectin (MCL), and Tulipa gesneriana lectin (TxLC-I) were purified in accordance with the method of Non-patent documents 2 to 4, respectively. Biotin-labeled Aleuria aurantia lectin (AAL), biotin-labeled Lens culinaris lectin (LCA), biotin-labeled Pisum sativum lectin (PSA), biotin-labeled Canavalia ensiformis lectin (ConA), biotin-labeled Sambucus sieboldiana lectin (SSA), biotin-labeled Ricinus communis lectin (RCA 120), biotin-labeled Erythrina cristagalli lectin (ECA), and biotin-labeled Phaseolus vulgaris lectin (PHA-E4). All of the biotin-labeled lectins were prepared with PBS to the concentration of 1 mg/mL, and diluted to an appropriate concentration in use.

The procedure of a pepsin treatment of glycoproteins is shown below.

### (1) Preparation of Reaction Solution

25 parts by mass of a 1 M glycine-hydrochloric acid buffer (pH 3.0) was added to 25 parts by mass of a 2 mg/mL glycoprotein solution.

### (2) Pepsin Reaction

1 part by mass (based on 100 parts by mass of the solution of (1)) of a pepsin solution (0.1 mg/mL 1 M glycine-hydrochloric acid buffer (pH 3.0)) was added to the solution of (1), stirred, and then allowed to stand at 37° C.

### (3) Termination of Reaction

After the pepsin reaction of 5 to 30 minutes, the solution and 25 parts by mass of a 1 M tris-hydrochloric acid buffer (pH 9.0) were mixed to terminate the reaction. These samples were taken as glycoprotein solutions after the pepsin treatment.

The procedure of an Actinase E treatment of glycoproteins is shown below.

### (1) Enzymatic Reaction

1 mass % of an Actinase E solution described above was diluted with 10 mM calcium chloride/100 mM tris-hydrochloric acid buffer (pH 7.8) described above so that the concentration was 0.00125 mass %, 42 μL of that diluted solution and 70 μL of an AFP-L3 solution at a concentration of 8 mg/mL were mixed and kept at 37° C. for 30 minutes.

### (2) Termination of Reaction

The reaction solution described above and 14 μL of 10 mM PMSF were mixed to terminate the proteolytic reaction, so as to obtain an Actinase E-treated AFP-L3 solution.

The procedure of a papain treatment of glycoproteins is shown below.

### (1) Enzymatic Reaction

5 μL of a papain solution at 2 mg/mL was added to 5 μL of an AFP-L3 solution at 6 mg/mL (dissolved in water), and kept at 37° C.

### (2) Termination of Reaction

At 30 minutes after the addition of the papain solution, the solution and 2 μL of antipain (from Sigma Aldrich Co. LLC.) were mixed to terminate the reaction, so as to a papain-treated AFP solution.

The procedure of the lectin ELISA (sandwich method) is shown below.

### (1) Antibody Immobilization

A solidified antibody, from which glycans were removed in accordance with the method described in Non-patent document 1, was diluted to 5 μg/mL with PBS, and 50 μL of this solution was added to each well of a 96-well microtiter plate (from Greiner Bio One International GmbH), allowed to stand at 4° C. for 16 hours, and then the additive solution was discarded.

### (2) Washing

250 μL, of 0.05% Tween/PBS was added to the well, and the additive solution was discarded.

### (3) Blocking

200 μL of BSA/PBS was added to the well and allowed to stand at 37° C. for an hour, and then the additive solution was discarded.

### (4) Washing

250 μL of 0.05% Tween/PBS was added to the well, and the additive solution was discarded. This manipulation was repeated three times in total.

### (5) Antigen-Antibody Reaction

50 μL of a glycoprotein solution diluted with BSA/PBS was added to the well and allowed to stand at room temperature for an hour, and then the additive solution was discarded.

### (6) Washing

250 μL of 0.05% Tween/PBS was added to the well, and the additive solution was discarded. This manipulation was repeated three times in total.

### (7) Lectin Reaction

A biotin-labeled lectin (1 mg/mL PBS) was diluted to an appropriate concentration with a BSA/PBS solution. 50 μL of this lectin solution was added to the well and allowed to stand at 4° C. for 30 minutes, and then the additive solution was discarded.

### (8) Washing

250 μL of 0.05% Tween/PBS was added to the well, and the additive solution was discarded. This manipulation was repeated three times in total.

### (9) Horseradish Peroxidase (HRP)-Labeled Streptavidin Reaction

An HRP-labeled streptavidin at 1 mg/mL: (from Funakoshi Co., Ltd.) was diluted with BSA/PBS (1 μg/mL). 50 μL of this solution was added to the well and allowed to stand at room temperature for 30 minutes, and then the additive solution was discarded.

### (10) Washing

250 μL of 0.05% Tween/PBS was added to the well, and the additive solution was discarded. This manipulation was repeated three times in total.

### (11) Coloring Reaction

50 μL of chromogenic substrate for HRP (TMB Microwell Peroxidase Substrate, from Funakoshi Co., Ltd.) was added to the well and allowed to stand at room temperature for 5 minutes.

### (12) Termination of Reaction

50 μL of 1 M phosphate was added to terminate the reaction.

Absorbance at 450 nm and 630 nm was measured using a plate reader (product name: POWERSCAN (registered trademark) HT, from BioTek Instruments, Inc.). A value obtained by subtracting the absorbance value at 630 nm from the absorbance value at 450 nm was taken as a detection value (signal: 5). A value obtained by subtracting the detection value in case of glycoproteins not being added (noise: N) from the detection value in case of glycoprotein being added (S) was taken as a reaction value (S-N).

As shown in Equation (1), a value obtained by subtracting the reaction value without the protease treatment (S-N)np from the reaction value with the protease treatment (S-N)p was taken as an increase amount Δ.

[Equation 1]

Increase amount Δ=(S-N)p−(S-N)np (1)

### [Examples 1 to 14] Detection of Pepsin-Treated AFP-L3 by Lectin Having Affinity with Fucose, Sialic Acid, Galactose, Mannose, Glucose Etc. (I)

A glycoprotein AFP-L3 was pepsin-treated for 30 minutes, and the resulting pepsin-treated AFP-L3 was detected by the lectin ELISA (sandwich method). In the detection test, the same manipulations as in the above-described sandwich ELISA method except for the followings:

For (1) Antibody immobilization, an anti-human AFP monoclonal antibody (mouse) (from Funakoshi Co., Ltd.) was used as the solidified antibody;

For (5) Antigen-antibody reaction, a pepsin-treated AFP-L3 (5,000 ng/mL) and an untreated AFP-L3 (5,000 ng/mL) were used as the glycoprotein solution;

For (7) Lectin reaction, the biotin-labeled lectins described in Table 1 were used, and an anti-AFP antibody (from Wako Japan) at 1 μg/mL was used instead of lectins;

For (9) HRP-labeled streptavidin reaction, when the anti-AFP antibody was used instead of lectins in (7), a solution prepared by diluting an HRP-labeled anti-rabbit IgG antibody solution (from Funakoshi Co., Ltd.) to 0.5 μg/mL with BSA/PBS was used instead of the HRP-labeled streptavidin.

The reaction values (S-N) with respect to the lectins or the antibody and the increase amount Δ of the pepsin-treated AFP-L3 and the untreated AFP-L3 are shown in Table 1.

TABLE 1

| | Lectin/Antibody | | | | |
|---|---|---|---|---|---|
| | | Concen- | | | Increase |
| | | tration | Reaction value (S-N) | | amount |
| | Name | (μg/mL) | (S-N)np | (S-N)p | Δ |
| Example 1 | PhoSL | 10 | 0.078 | 1.309 | 1.231 |
| Example 2 | PhoSL | 10 | 0.010 | 0.704 | 0.694 |
| | peptide | | | | |
| Example 3 | AAL | 10 | −0.002 | 0.473 | 0.475 |
| Example 4 | AOL | 10 | −0.013 | 0.218 | 0.231 |
| Example 5 | LCA | 10 | 0.097 | 1.614 | 1.517 |
| Example 6 | PSA | 1 | 0.770 | 1.277 | 0.507 |
| Example 7 | ConA | 10 | 0.237 | 0.582 | 0.345 |
| Example 8 | SNA-I | 1 | 0.013 | 0.160 | 0.147 |
| Example 9 | SSA | 10 | 0.040 | 0.699 | 0.659 |
| Example 10 | RCA120 | 1 | 0.232 | 0.585 | 0.353 |
| Example 11 | ECA | 10 | 0.005 | 0.016 | 0.011 |
| Example 12 | PHA-E4 | 10 | 0.033 | 0.194 | 0.161 |
| Example 13 | MCL | 10 | 0.004 | 0.023 | 0.019 |
| Example 14 | TxLCI | 10 | 0.008 | 0.349 | 0.341 |
| Comparative | Anti-AFP | 1 | 1.142 | 1.023 | −0.119 |
| Example 1 | antibody | | | | |

In all Examples, the reaction values between the protease-treated AFP-L3 and lectins were increased from the reaction values between the protease-untreated AFP-L3 and lectins. On the other hand, in Comparative Example 1 using the antibody, the reaction value was not increased by the protease treatment. Therefore, it was confirmed that the increase of the reaction values of the protease-treated glycoprotein AFP-L3 with lectins (i.e., improvement of the detection sensitivity of the glycoprotein) was not due to the increase of the binding amount of the glycoprotein to the immobilized antibody.

### [Examples 15 to 21] Detection of Pepsin-Treated AFP by Lectin Having Affinity with Sialic Acid, Galactose, Mannose, Glucose Etc

A glycoprotein AFP was pepsin-treated for 30 minutes, and the resulting pepsin-treated AFP was detected by the lectin ELISA (sandwich method). The detection test was carried out with the same procedure as that of Examples 7 to 13 and Comparative Example 1 except for replacing the glycoprotein AFP-L3 by AF. The reaction values (S-N) with respect to each of the lectins and the increase amount Δ of the pepsin-treated AFP and the untreated AFP are shown in Table 2.

TABLE 2

| | Lectin/antibody | | | | |
|---|---|---|---|---|---|
| | | Concen- | | | Increase |
| | | tration | Reaction value (S-N) | | amount |
| | Name | (μg/mL) | (S-N)np | (S-N)p | Δ |
| Example 15 | ConA | 10 | 0.306 | 0.539 | 0.233 |
| Example 16 | SNA-I | 1 | 0.009 | 0.075 | 0.066 |
| Example 17 | SSA | 10 | 0.018 | 0.251 | 0.233 |
| Example 18 | RCA120 | 1 | 0.176 | 0.432 | 0.256 |
| Example 19 | ECA | 10 | 0.031 | 0.052 | 0.021 |
| Example 20 | PHA-E4 | 10 | 0.043 | 0.428 | 0.385 |
| Example 21 | MCL | 10 | 0.012 | 0.031 | 0.019 |
| Comparative | Anti-AFP | 1 | 1.212 | 1.052 | −0.160 |
| Example 2 | antibody | | | | |

With reference to Table 2, in all Examples, the reaction values between the protease-treated AFP and lectins were increased from the reaction values between the protease-untreated AFP and lectins. On the other hand, in Comparative Example 2 using the antibody, the reaction value was not increased by the protease treatment. Therefore, it was confirmed that the increase of the reaction values between the glycoprotein and lectins, i.e., improvement of the detection sensitivity of the glycoprotein was not due to the increase of the AFP binding amount of the glycoprotein to the immobilized antibody.

### [Example 22] Detection of Actinase E-Treated AFP-L3 by Lectin Having Affinity with Fucose

The protease treatment of glycoproteins in Example 1 is changed from the pepsin treatment to the Actinase E-treatment. Specifically, the same manipulations were carried out as those in Example 1 except for the followings:

For (5) Antigen-antibody reaction, an Actinase E-treated AFP-L3 (400 ng/mL) and an untreated AFP-L3 (400 ng/mL) were used as the glycoprotein solution;

For (7) Lectin reaction, a biotin-labeled PhoSL (0.5 μg/mL) was used

The lectin reaction values (S-N) of the protease-treated AFP-L3 and the untreated AFP-L3, and the increase amount Δ of the reaction values due to the protease treatment are shown in Table 3.

TABLE 3

| | Increase |
|---|---|
| Reaction value (S-N) | amount |

| | Lectin | (S-N)np | (S-N)p | Δ |
|---|---|---|---|---|
| Example 22 | PhoSL | 0.036 | 0.117 | 0.081 |

With reference to Table 3, it was found that the reaction values between the AFP-L3 and lectins were increased even if the Actinase E, which was a proteolytic enzyme different from pepsin, was used for the protease treatment.

### [Example 3] Detection of Papain-Treated AFP-L3 by Lectin Having Affinity with Fucose

The protease treatment of glycoproteins in Example 1 is changed from the pepsin treatment to the papain-treatment. Specifically, the same manipulations were carried out as those in Example 1 except for the followings:

For (1) Antibody immobilization, an anti-human AFP monoclonal antibody (mouse) (from Funakoshi Co., Ltd.) was used as the solidified antibody;

For (5) Antigen-antibody reaction, a papain-treated AFP-L3 (1000 ng/mL) and an untreated AFP-L3 (1000 ng/mL) were used as the glycoprotein solution;

For (7) Lectin reaction, a biotin-labeled PhoSL (0.5 μg/mL) was used.

The lectin reaction values (S-N) of the protease-treated AFP-L3 and the untreated AFP-L3, and the increase amount Δ of the reaction values due to the protease treatment are shown in Table 4.

TABLE 4

| | |
|---|---|
| | Increase |
| Reaction value (S-N) | amount |

| | Lectin | (S-N)np | (S-N)p | Δ |
|---|---|---|---|---|
| Example 23 | PhoSL | 0.005 | 0.100 | 0.095 |

With reference to Table 4, it was found that the reaction values between the AFP-L3 and lectins were increased even if the papain, which was a proteolytic enzyme different from pepsin, was used for the protease treatment.

### [Example 24] Detection of Pepsin-Treated Transferrin (′IT) by Lectin Having Affinity with Sialic Acid

A glycoprotein TF was pepsin-treated for 5 minutes, and the resulting pepsin-treated TF was detected by the lectin ELISA (sandwich method). In the detection test, the same manipulations as in the above-described sandwich ELISA method except for the followings:

For (1) Antibody immobilization, an anti-human transferrin polyclonal antibody (rabbit) (from Funakoshi Co., Ltd.) was used as the immobilized antibody;

For (5) Antigen-antibody reaction, a pepsin-treated TF solution (1 μg/mL) and an untreated TF solution (1 μg/mL) were used as the glycoprotein solution;

For (7) Lectin reaction, the biotin-labeled SSA (1 μg/mL) described in Table 1 were used. And an anti-human transferrin polyclonal antibody (mouse) (from Cosmo Bio Co., Ltd.) at 0.2 μg/mL was used instead of lectins;

For (9) Horseradish peroxidase (HRP)-labeled streptavidin reaction, when the anti-TF antibody was used instead of lectins in (7), a solution prepared by diluting an HRP-labeled anti-mouse IgG antibody solution (from Invitrogen Co., Ltd.) to 0.5 μg/mL with BSA/PBS was used instead of the HRP-labeled streptavidin.

The reaction values (S-N) with respect to the lectins or the antibody and the increase amount Δ of the pepsin-treated TF and the untreated IF are shown in Table 5.

TABLE 5

| | | | | Increase |
|---|---|---|---|---|
| | | Reaction value (S-N) | | amount |
| | Lectin/Antibody | (S-N)np | (S-N)p | Δ |
| Example 24 | SSA | 0.120 | 0.387 | 0.267 |
| Comparative | Anti-TF antibody | 1.442 | 1.302 | −0.140 |
| Example 3 | | | | |

With reference to Table 5, it was found that the reaction values could be increased by the pepsin treatment even if the transferrin was detected by a sialic acid specific lectin SSA. On the other hand, when the transferrin was detected by the anti-IF antibody, the reaction value did not increase due to the pepsin treatment, whereby it was found that the increase of the lectin reaction values was not due to the increase of the transferrin amount captured by the immobilized antibody

### [Examples 25 to 26] Detection of Pepsin-Treated Immunoglobulin G (IgG) by Lectin Having Affinity with Sialic Acid

A glycoprotein IgG was pepsin-treated for 5 minutes, and the resulting pepsin-treated IgG was detected by the lectin ELISA (sandwich method). In the test, the same manipulations as in the above-described sandwich ELISA method except for the followings:

For (1) Antibody immobilization; an anti-human immunoglobulin polyclonal antibody (goat) (from Funakoshi Co., Ltd.) was used as the solidified antibody;

For (5) Antigen-antibody reaction, a pepsin-treated IgG solution (1 μg/mL) and an untreated IgG solution (1 μg/mL) were used as the glycoprotein solution;

For (7) Lectin reaction, a biotin-labeled. PhoSL and a biotin-labeled AAL (both 1 μg/mL) were used. And the anti-human immunoglobulin polyclonal antibody (goat) at 0.2 μg/mL was used instead of lectins;

For (9) Horseradish peroxidase (HRP)-labeled streptavidin reaction, when the anti-human immunoglobulin polyclonal antibody (goat) was used instead of lectins in (7), a solution prepared by diluting an HRP-labeled anti-goat IgG antibody solution (from Cosmo Bio Co., Ltd.) to 0.5 μg/mL with BSA/PBS was used instead of the HRP-labeled streptavidin.

The reaction values (S-N) with respect to the lectins or the antibody and the increase amount Δ of the pepsin-treated. IgG and the untreated IgG are shown in Table 6.

TABLE 6

| | | | | Increase |
|---|---|---|---|---|
| | | Reaction value (S-N) | | amount |
| | Lectin/Antibody | (S-N)np | (S-N)p | Δ |
| Example 25 | PhoSL | 0.749 | 1.245 | 0.496 |
| Example 26 | AAL | 0.063 | 0.101 | 0.038 |
| Comparative | Anti-IgG | 1.010 | 0.894 | −0.116 |
| Example 4 | antibody | | | |

With reference to Table 6, it was found that the reaction values could be increased by the pepsin treatment even if the IgG was detected by the fucose specific lectins such as PhoSL or AAL. On the other hand, when the IgG was detected by the anti-IgG antibody, the reaction value did not increase due to the pepsin treatment, whereby it was found that the increase of the lectin reaction values was not due to the increase of the IgG amount captured by the immobilized antibody.

### [Examples 27 to 34] Detection of Pepsin-Treated AFP-L3 by Lectin Having Affinity with Fucose or Sialic Acid (II)

A pepsin treatment was carried out by using serum to which an AFP-L3 was added, and the detection test of the AFP-L3 was carried out by the sandwich ELISA method. The procedure of the pepsin treatment of the serum solution of the AFP-L3 is shown below.

### (1) Preparation of AFP-L3 Solution

Eight μL, of an AFL-L3 solution at 50 μg/mL was added to each of 992 μL it of human pooled serum (100% serum, KAC Co., Ltd.), and 992 μL of 10% serum prepared by diluting this 100% serum by ten times with PBS to prepare AFP-L3/100% serum and AFP-L3/10% serum (for both, AFP-L3 concentration: 400 ng/mL).

### (2) Pepsin Reaction

Twenty-two μL of a pepsin/1 M glycine-hydrochloric acid buffer (pH 3.0) was added to 55 μL of the AFP-L3/100% serum, stirred, and then allowed to stand at 25° C. 10 μL of a pepsin/1 M glycine-hydrochloric acid buffer (pH 3.0) was added to 55 μL of the AFP-L3/10% serum, stirred, and then allowed to stand at 25° C.

### (3) Termination of Reaction

After 30 minutes of the addition of the pepsin solution, 33 μL of a 0.33M tris-hydrochloric acid buffer (pH 9.0) was added to each of the serum to terminate the reaction, so as to obtain pepsin-treated AFP-L3/100% serum and pepsin-treated AFP-L3/10% serum.

In the detection test by lectins, the same manipulations as in the above-described sandwich ELISA method except for the followings:

For (1) Antibody immobilization, an anti-human AFP monoclonal antibody (mouse) (from Funakoshi Co., Ltd.) was used as the solidified antibody;

For (5) Antigen-antibody reaction, the pepsin-treated AFP-L3/100% serum, the pepsin-treated AFP-L3/10% serum, the untreated AFP-L3/100% serum, and the untreated AFP-L3/10% serum (for all, AFP-L3 concentration: 0.2 μg/mL) were used as the glycoprotein solution;

For (7) Lectin reaction, a biotin-labeled PhoSL, a biotin-labeled AAL, a biotin-labeled SNA-1 and a biotin-labeled SSA (all at 0.5 μg/mL) were used.

The reaction values (S-N) with respect to the lectins and the increase amount Δ of the pepsin-treated AFP-L3/100% serum, the pepsin-treated AFP-L3/10% serum, the untreated AFP-L3/100% serum, and the untreated AFP-L3/10% serum are shown in Table 7.

TABLE 7

| | | | | | Increase |
|---|---|---|---|---|---|
| | | | Reaction value (S-N) | | amount |
| | Glycoprotein | Lectin | (S-N)np | (S-N)p | Δ |
| Example 27 | AFP-L3/10% | PhoSL | 0.042 | 0.433 | 0.391 |
| Example 28 | serum | AAL | 0.066 | 0.440 | 0.374 |
| Example 29 | | SNA-I | 0.153 | 0.472 | 0.319 |
| Example 30 | | SSA | 0.124 | 0.278 | 0.154 |
| Example 31 | AFP-L3/100% | PhoSL | 0.054 | 0.385 | 0.331 |
| Example 32 | serum | AAL | 0.172 | 0.355 | 0.183 |
| Example 33 | | SNA-I | 0.214 | 0.450 | 0.236 |
| Example 34 | | SSA | 0.203 | 0.339 | 0.136 |

With reference to Table 7, it was found that the reaction values could be increased by the pepsin treatment even if the AFP-L3 was detected by the fucose specific lectin PhoSL or AAL in the presence of the serum. With reference to Table 7, it was confirmed that the reaction values could be similarly increased by the pepsin treatment even if the AFP-L3 was detected by the sialic acid specific SNA-1 or SSA. Therefore, it could be confirmed that the reaction values could be increased even for the glycoproteins in the human serum.

### [Example 35] Detection of Pepsin-Treated AFP-L3 by Lectin Having Affinity with Fucose

A pepsin treatment of serum to which an AFP-L3 was added with varying time, and the detection test of the AFP-L3 was carried out on each treatment sample by the sandwich ELISA method. The procedure of the pepsin treatment was the same as the procedure of the pepsin treatment of the AFP-L3/100% serum (AFP-L3: 400 ng/mL) in Example 31 except for that the time between the addition of the pepsin and the termination of the reaction was varied from 30 minutes to 5, 10, 15, 30, 45 or 60 minutes.

The detection test by lectins was carried out with the same manipulations as in Example 31.

The reaction values (S-N) with respect to the lectins and the increase amount Δ of the pepsin-treated AFP-L3/100% serum and the untreated AFP-L3/100% serum are shown in Table 8.

TABLE 8

| | | Pepsin treatment | | Increase |
|---|---|---|---|---|
| | | time | Reaction | amount |
| | lectin | (min.) | value (S-N) | Δ |
| Control | PhoSL | Untreated | 0.054 | — |
| Example 35 | | 5 | 0.295 | 0.241 |
| Example 36 | | 10 | 0.389 | 0.335 |
| Example 37 | | 15 | 0.417 | 0.363 |
| Example 31 | | 30 | 0.385 | 0.331 |
| Example 38 | | 45 | 0.418 | 0.364 |
| Example 39 | | 60 | 0.415 | 0.361 |

With reference to Table 8, the increase amount of the reaction value was increased to 0.241 at 5 minutes after the pepsin treatment. The increase amount by the pepsin treatment for 10 to 60 minutes was substantially constant at 0.331 to 0.364. It was found that the increasing effect could be obtained at the reaction time of from 5 minutes to at least 60 minutes.

### [Example 40] Detection of Pepsin-Treated AFP-L3 by HPLC-Lectin Column

A glycoprotein was pepsin-treated for 30 minutes by using an AFP-L3, and the binding test of a pepsin-treated AFP-L3 was carried out by an HPLC-PhoSL column. The specific procedure thereof is shown below.

### (1) Production of HPLC-PhoSL Column

PhoSL was immobilized into an activation hard gel (from J-OIL MILLS. Inc.) in accordance with the attached manual, and then filled into a stainless column (150 mm×4.6 mm I.D.).

### (2) Analysis on Pepsin-Treated AFP-L3 by HPLC-PhoSL Column

### 1. Preparation of Buffer for HPLC

Buffer A (50 mM ammonium acetate): Water was added to 50 mL of 1 M ammonium acetate (from Wako Pure Chemical Corp.) to 1000 mL,

Buffer B (0.2 N ammonia): Water was added to 7.55 mL of a 25% ammonia solution (from Sigma-Aldrich Co. LLC.) to 500 mL.

### 2. HPLC Analysis

Using an HPLC analysis apparatus (system: LC-8020, pump: DP-8020, both from Tosoh Corporation), 100 μL of a solution prepared by diluting the pepsin-treated AFP-L3 or the pepsin-untreated AFP-L3 by ten times with the buffer A was injected. The buffer A was allowed to flow for 10 minutes to obtain an unadsorption peak, and then the buffer B was allowed to flow for 10 minutes to obtain an adsorption-elution peak. The flow rate was 0.5 mL/min, and the detection was carried out at UV 280 nm (UV-2080, from Tosoh Corporation).

The non-adsorbed peak area and the adsorbed-elution peak area were measured when the untreated and pepsin-treated AFP-L3 were analyzed by HPLC-PhoSL. The binding rate was calculated in accordance with the following equation:

$$\begin{matrix} [ Equation 2 ] & \\ Binding Rate ( \% ) = \frac{Adsorbed \text{-} elution peak area}{Non \text{-} adsorbed peak area + Adsorbed \text{-} elution peak area} \times 100 & ( 2 )\end{matrix}$$

The non-adsorbed peak area, the adsorption-elution peak area and the binding rate (%) of the pepsin-treated AFP-L3 by HPLC-PhoSL are shown in Table 9.

TABLE 9

| | | | | Adsorbed- | |
|---|---|---|---|---|---|
| | | | | elution | |
| | | | Non-adsorbed | peak | Binding |
| | | Pepsin | peak area | area | rate |
| | Lectin | treatment | (mVSec) | (mVSec) | (%) |
| Control | PhoSL | Untreated | 2879 | 243 | 7.8 |
| Example 40 | | Pepsin | 1436 | 574 | 29 |
| | | treated | | | |

With reference to Table 9, it was found that the detection value (binding rate) of the pepsin-treated AFP-L3 by HPLC-PhoSL was increased from that of the untreated AFP-L3.

### [Example 41] Detection of Pepsin-Treated AFP-L3 by Lectin Having Affinity with Fucose (IV)

Serum to which an AFP-L3 was added was pepsin-treated with varying the pepsin concentration and time, and the detection test of the AFP-L3 was carried out on each treatment sample by the sandwich ELISA method. The pepsin treatment was carried out as follows.

### (1) Preparation of AFP-L3 Solution

An AFL-L3 solution was added to human pooled serum to prepare AFL-L3/100% serum (AFP-L3 concentration: 200 ng/mL).

### (2) Preparation of Reagent

### [1.2 M Glycine-Hydrochloric Acid Buffer (pH 3.25)]

90.08 g of glycine was dissolved in 900 mL of water, to which 6N hydrochloric acid was added to adjust the pH level to 3.25 with a pH meter, and in addition, the volume was further increased to 1000 mL with water.

### [0.1% BSA/1.2 M Glycine-Hydrochloric Acid Buffer (pH 3.25)]

It was prepared by dissolving 0.1 g of bovine serum albumin (BSA, from Sigma-Aldrich Co, LLC) in 100 mL of 1.2 M glycine-hydrochloric acid buffer (pH 3.25).

### (3) Pepsin Reaction

30 μL of a pepsin dissolved in 0.1% BSA/1.2 M glycine-hydrochloric acid buffer (pH 3.25) was added to 60 μL of the AFP-L3/100% serum, stirred, and then allowed to stand at 25° C.

### (4) Termination of Reaction

After 5, 7, 9, 11, 13 or 15 minutes, 30 μL of a 0.33M tris-hydrochloric acid buffer (pH 9.0) was added to terminate the reaction.

In the detection test by lectins, the same manipulations as in the above-described sandwich ELISA method except for the followings:

For (1) Antibody immobilization, an anti-human AFP monoclonal antibody (mouse) (from Funakoshi Co., Ltd.) was used as the solidified antibody;

For (5) Antigen-antibody reaction, the pepsin-treated AFP-L3/100% serum and the untreated AFP-L3/100% serum were used as the glycoprotein solution (for both, AFP-L3 concentration: 0.1 μg/mL);

For (7) Lectin reaction, the biotin-labeled PhoSL (0.5 μg/mL) was used.

The detection values with respect to the lectins of the pepsin-treated AFP-L3/100% serum and the untreated AFP-L3/100% serum are shown in Table 10.

TABLE 10

| | | Pepsin treatment time (min.) | | | | | | |
|---|---|---|---|---|---|---|---|---|
| | | Untreated | 5 | 7 | 9 | 11 | 13 | 15 |
| Concen- | 0.47 | 0.222 | 0.880 | 1.030 | 1.016 | 1.012 | 0.998 | 1.005 |
| tration | 0.63 | 0.222 | 1.011 | 1.033 | 0.975 | 0.993 | 0.99 | 0.984 |
| of added | 0.78 | 0.222 | 0.999 | 1.067 | 0.974 | 1.012 | 0.963 | 0.923 |
| pepsin | 0.94 | 0.222 | 1.041 | 0.989 | 0.977 | 0.941 | 0.913 | 0.835 |
| (ng/mL) | 1.1 | 0.222 | 1.016 | 0.986 | 0.939 | 0.904 | 0.885 | 0.843 |

As shown in Table 10, it was found that the reaction values could be sufficiently enhanced even in a short time by changing the concentration of the pepsin.

### SEQUENCE LISTING

1. A method for detecting glycoproteins, comprising steps of:
subjecting a sample containing the glycoprotein to a protease treatment;
allowing the protease-treated glycoprotein to react with a sugar-binding compound having affinity with a glycan contained in the glycoprotein in order to obtain a reactant between the glycoprotein and the sugar-binding compound; and
detecting the reactant.

2. The method for detecting glycoproteins according to claim 1, wherein
the protease treatment is a pepsin treatment, a papain treatment or an actinase treatment.

3. The method for detecting glycoproteins according to claim 1, wherein
the sample is serum.

4. The method for detecting glycoproteins according to claim 1, wherein
the sugar-binding compound is a sugar-binding protein.

5. The method for detecting glycoproteins according to claim 1, wherein
the sugar-binding compound has affinity with at least one selected from the group consisting of fucose, sialic acid, mannose, glucose, galactose, N-acetylglucosamine and N-acetylgalactosamine.

6. The method for detecting glycoproteins according to claim 1, wherein
the glycoprotein is immobilized to a support.

7. The method for detecting glycoproteins according to claim 6, wherein
the glycoprotein is immobilized to the support via an antibody of the glycoprotein.

8. The method for detecting glycoproteins according to claim 1, wherein
the sugar-binding compound and/or a probe for detecting the sugar-binding compound are labeled.

9. The method for detecting glycoproteins according to claim 1, wherein
the glycan is a glycan of complex type, a glycan of high mannose type or an O-linked glycan.

10. The method for detecting glycoproteins according to claim 1, wherein
the glycoprotein is selected from a group consisting of haptoglobin, fucosylated haptoglobin, transferrin, γ-glutamyltranspeptidase, immunoglobulin G, immunoglobulin A, immunoglobulin M, α1-acidic glycoprotein, α-fetoprotein, fucosylated α-fetoprotein, fibrinogen, human placenta chorionic gonadotropin, carcinoembryonic antigen, prostate-specific antigen, fucosylated prostate-specific antigen, thyroglobulin, fetuin and asialofetuin.


SEQUENCE LISTING (1 sequence)

SEQ ID NO: 1 - protein, 40 aa, Artificial sequence
  PhoSL synthesized by the method described in Non-patent document 1
APVPVTKLVCDGDTYKCTAYLDFGDGRWVAQWDTNVFHTG